(19) United States
(12) Patent Application Publication
(10) Pub. No.: US 2026/0151226 A1
(43) Pub. Date: Jun. 4, 2026

(54) PROSTHETIC HEART VALVES FOR DEPLOYMENT TO A MITRAL VALVE

(71) Applicant: Edwards Lifesciences Corporation, Irvine, CA (US)

(72) Inventors: Abdorruhman M. Yousef, Santa Ana, CA (US); Adeeb Saiduddin, Lake Forest, CA (US); Munir Hicham Eltal, Irvine, CA (US); Anthony Michael Romero, Corona, CA (US)

(73) Assignee: Edwards Lifesciences Corporation, Irvine, CA (US)

(21) Appl. No.: 19/420,449

(22) Filed: Dec. 15, 2025

Related U.S. Application Data
(63) Continuation of application No. PCT/US2024/035717, filed on Jun. 27, 2024.
(60) Provisional application No. 63/523,693, filed on Jun. 28, 2023.

(51) Int. Cl. A61F 2/24; A61F 2/848

(52) U.S. Cl. CPC A61F 2/2418; A61F 2002/8483; A61F 2210/0014; A61F 2230/0004; A61F 2250/0069

(57) ABSTRACT

Improved features of medical devices. Implants that may include self-expanding portions and portions that are expandable with an inflatable body. A portion expandable with an inflatable body may dock with a portion that is self-expanding. A portion expandable with an inflatable body may be coupled to a portion that is self-expanding. Delivery apparatuses for deployment of both a self-expanding portion and a portion expandable with an inflatable body may be disclosed.

### CROSS-REFERENCE TO RELATED APPLICATIONS

This application is a continuation of International Application No. PCT/US2024/035717, filed Jun. 27, 2024, which designates the United States and was published in English by the International Bureau on Jan. 2, 2025 as WO2025/006678, which claims the benefit of U.S. Provisional Application No. 63/523,693, filed Jun. 28, 2023, the entire contents of which are incorporated herein by reference.

### BACKGROUND

### Field

Certain examples disclosed herein relate generally to implantable medical devices and may relate to implantable prosthetic valves such as those which may be used in place of native heart valves.

### Background

A variety of maladies may affect an individual's body. Such maladies may be of the individual's heart, and may include maladies of the individual's native heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a patient's heart. Prosthetic implants, such as prosthetic valves, may be provided to replace or repair a portion of a patient's heart. Prosthetic aortic, mitral, tricuspid, and even pulmonary valves may be provided.

Implants may be deployed to the desired portion of the patient's body percutaneously, in a minimally invasive manner. Such deployment may occur transcatheter, in which a catheter may be deployed through the vasculature of an individual.

Deployment to a mitral or tricuspid valve may pose challenges. The shape of the mitral or tricuspid valve annulus may be irregular. The presence of chordae may need to be addressed. Variations in the shape of the mitral or tricuspid valve among different patients may be significant.

### SUMMARY

Features of examples disclosed herein may be directed to improved features of medical devices. The features may include implants that may include self-expanding portions and portions that are expandable with an inflatable body. In examples, a portion expandable with an inflatable body may dock with a portion that is self-expanding. In examples, one or more couplers may be utilized that may couple the portion expandable with an inflatable body to the portion that is self-expanding. Variations may be provided.

Aspects of the present disclosure may include a system for deployment to a native heart valve.

The system may include a self-expanding stent configured to be deployed to the native heart valve and including a proximal portion, a distal portion, and a central portion having a smaller diameter than the proximal portion and the distal portion, the proximal portion comprising a flange extending radially outward from the central portion and having a conical shape from the central portion to a proximal end of the self-expanding stent, the self-expanding stent surrounding an interior cavity.

The system may include a valve body configured to be expanded with an inflatable body and configured to be positioned within the interior cavity of the self-expanding stent and couple to the central portion, the valve body including one or more prosthetic valve leaflets.

Aspects of the present disclosure may include a delivery system for deployment of a prosthetic system to a native heart valve.

The delivery system may include an elongate shaft.

The elongate shaft may include a first implant retention area comprising an inflatable body for a first implant to be positioned upon, the inflatable body configured to expand to deploy the first implant to the native heart valve from the elongate shaft.

The elongate shaft may include a second implant retention area comprising a retaining body configured to retain a second self-expanding implant to the elongate shaft in a compressed configuration, the retaining body configured to release from the second self-expanding implant to allow the second self-expanding implant to deploy to the native heart valve from the elongate shaft.

Aspects of the present disclosure may include a method.

The method may include deploying a prosthetic system to a native heart valve.

The prosthetic system may include a self-expanding stent including a proximal portion, a distal portion, and a central portion having a smaller diameter than the proximal portion and the distal portion, the proximal portion comprising a flange extending radially outward from the central portion and having a conical shape from the central portion to a proximal end of the self-expanding stent, the self-expanding stent surrounding an interior cavity.

The prosthetic system may include a valve body configured to be expanded with an inflatable body and configured to be positioned within the interior cavity of the self-expanding stent and couple to the central portion, the valve body including one or more prosthetic valve leaflets.

Aspects of the present disclosure may include a method.

The method may include deploying a prosthetic system to a native heart valve utilizing a delivery system, the prosthetic system including a first implant and a second self-expanding implant.

The delivery system may include an elongate shaft having: a first implant retention area comprising an inflatable body for the first implant to be positioned upon, the inflatable body configured to expand to deploy the first implant to the native heart valve from the elongate shaft.

The delivery system may include a second implant retention area comprising a retaining body configured to retain the second self-expanding implant to the elongate shaft in a compressed configuration, the retaining body configured to release from the second self-expanding implant to allow the second self-expanding implant to deploy to the native heart valve from the elongate shaft.

### BRIEF DESCRIPTION OF THE DRAWINGS

Features and advantages of the systems, devices, and methods as disclosed herein will become appreciated as the same become better understood with reference to the specification, claims, and appended drawings wherein:

FIG. 1 illustrates a perspective view of an implantable valve body.

FIG. 2 illustrates a top view of the implantable valve body shown in FIG. 1.

FIG. 3 illustrates a top view of the implantable valve body shown in FIG. 1, with prosthetic valve leaflets moved from the position shown in FIG. 2.

FIG. 4 illustrates a side perspective view of a stent.

FIG. 5 illustrates a perspective view of a stent frame of a stent.

FIG. 6 illustrates a top view of the stent frame shown in FIG. 5.

FIG. 7 illustrates a perspective view of a stent frame of a stent.

FIG. 8 illustrates a side view of the stent frame shown in FIG. 7.

FIG. 9 illustrates a top view of the stent frame shown in FIG. 7.

FIG. 10 illustrates a side view of a stent frame.

FIG. 11 illustrates a side cross sectional schematic view of the stent shown in FIG. 4.

FIG. 12 illustrates a perspective view of the stent frame shown in FIG. 5 with a valve body docked to the stent.

FIG. 13 illustrates a side cross sectional schematic view of the valve body docked to a stent.

FIG. 14 illustrates a side view of a delivery system.

FIG. 15A illustrates a side view of the distal end portion of the delivery system shown in FIG. 14.

FIG. 15B illustrates a side view of the distal end portion of the delivery system shown in FIG. 14.

FIG. 15C illustrates a side view of the distal end portion of the delivery system shown in FIG. 14.

FIG. 16 illustrates a side cross sectional schematic view of the delivery system shown in FIG. 14.

FIG. 17 illustrates a schematic view of a delivery system approaching an implantation site.

FIG. 18A illustrates a side cross sectional schematic view of a delivery system approaching an implantation site.

FIG. 18B illustrates a side cross sectional schematic view of a self-expanding stent deployed to an implantation site.

FIG. 18C illustrates a side cross sectional schematic view of an inflatable body expanding a valve body to dock the valve body with the stent.

FIG. 18D illustrates a side cross sectional schematic view of a valve body docked to a stent.

FIG. 19 illustrates a side cross sectional schematic view of a distal end portion of a delivery system.

FIG. 20 illustrates a side cross sectional schematic view of a distal end portion of a delivery system.

FIG. 21 illustrates a side cross sectional schematic view of a distal end portion of a delivery system.

FIG. 22 illustrates a side cross sectional schematic view of a distal end portion of a delivery system.

FIG. 23 illustrates a side cross sectional schematic view of a distal end portion of a delivery system.

FIG. 24 illustrates a side cross sectional schematic view of a valve body coupled to a stent.

FIG. 25 illustrates a side cross sectional schematic view of a valve body coupled to a stent.

### DETAILED DESCRIPTION

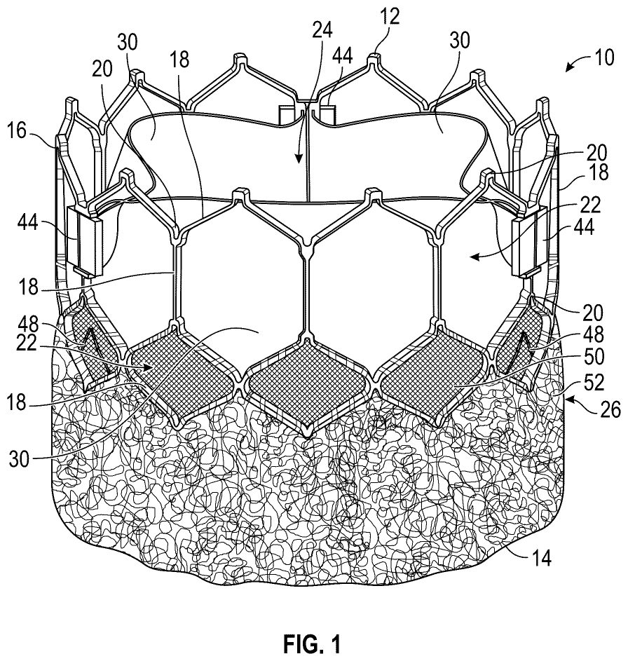

FIG. 1 illustrates a perspective view of a valve body 10. The valve body 10 may have a distal end 12, a proximal end 14, and a length between the proximal end 14 and the distal end 12. The distal end 12 may comprise an outflow end of the valve body 10, and the proximal end 14 may comprise an inflow end of the valve body 10.

The valve body 10 may have a cylindrical shape as shown in FIG. 1 or may have another shape as desired.

The valve body 10 may include a stent frame 16 in examples. The stent frame 16 may include a plurality of struts 18 that may join together at junctures 20 and may have spaces 22 between the struts 18. The spaces 22 may comprise openings of the frame 16 that may allow fluid flow therethrough in examples. The spaces 22 may be configured to reduce the overall weight of the stent frame 16, and also allow the stent frame 16 to be compressed to reduce a diameter of the stent frame 16 and be expanded to increase a diameter of the stent frame 16. The stent frame 16 may have a cylindrical shape. The configuration of the stent frame may vary in other examples.

The plurality of struts 18 may form a lattice structure for the stent frame 16. The lattice structure may include cells comprising the struts 18 bounding one of the spaces 22. The cells may repeat to form the lattice structure. The cells may have a diamond shape as shown in FIG. 1 or may have another shape as desired.

The stent frame 16 may be configured to allow the valve body 10 to be collapsible and expandable, with the stent frame 16 being compressed (or crimped) to move to a collapsed (or undeployed or unexpanded) state and being expanded to move to an expanded (or deployed) state. The stent frame 16 may be configured to be radially compressed and axially lengthened while being radially compressed.

The struts 18 may be configured such that as the stent frame 16 is compressed to reduce a diameter of the stent frame 16, the length of the stent frame 16 may increase. Also, as the stent frame 16 is expanded to increase the diameter of the stent frame 16, the length of the stent frame 16 may decrease. The stent frame 16 may be compressed in a variety of manners, including use of a crimping device, and may be expanded with an inflatable body such as a balloon in examples.

The plurality of struts 18 may be configured to move closer together to allow the stent frame 16 to move to the collapsed state. The width of the spaces 22 between the struts 18 may be reduced as the stent frame 16 is moved to the collapsed (or undeployed or unexpanded) state, with the length of the spaces 22 increasing. The struts 18 of the stent frame 16 may be configured to circumferentially move away from each other to move to the expanded state. The width of the spaces 22 between the struts 18 may be increased as the stent frame 16 is moved to the expanded (or deployed) state, with the length of the spaces 22 decreasing.

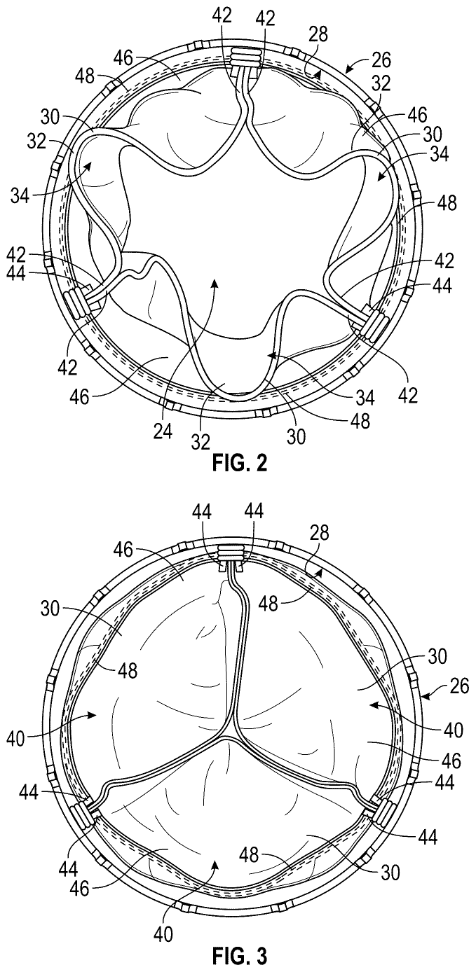

The valve body 10 may surround a flow channel 24 that may allow for flow of fluid (e.g., blood or another fluid) through the valve body 10. The valve body 10 may include an outer surface 26 that may face outward from the valve body 10 and may include an inner surface 28 (as marked in FIGS. 2 and 3) that faces opposite the outer surface 26 and faces the flow channel 24. The outer surface 26 may comprise an anchoring or docking surface that may be utilized to anchor or dock the valve body 10 as desired. The outer surface 26 may apply a force radially outward to anchor or dock the valve body 10.

The valve body 10 may include one or more prosthetic valve leaflets 30. The prosthetic valve leaflets 30 may be positioned within the flow channel 24 and may extend inward from the inner surface 28 of the valve body 10. The one or more prosthetic valve leaflets 30 may be configured to move away from each other to move to an open position (as shown in FIG. 2) and may be configured to move towards each other to move to a closed position (as shown in FIG. 3). Each prosthetic valve leaflet 30 may each include an upper end portion 32 (marked in FIG. 2), and the upper end portions 32 of the valve leaflets 30 may be configured to contact each other to close the flow channel 24 of the valve body 10 when the leaflets 30 are in the closed position. The upper end portions 32 are configured to move away from each other to open the flow channel 24 of the valve body 10 when the prosthetic valve leaflets 30 are in the open position (as shown in FIG. 2). The prosthetic valve leaflets 30 may move back and forth between open and closed positions or states or configurations to replicate the motion of a native valve.

Each prosthetic valve leaflet 30 may include an interior surface 34 (marked in FIG. 2) configured to face towards the flow channel 24 of the valve body 10. Each valve leaflet 30 may include an exterior surface 40 (marked in FIG. 3) facing opposite the interior surface 34 and facing away from the flow channel 24 of the valve body 10. Portions of the interior surface 34 of respective leaflets 30 may contact each other when the leaflets 30 move to the closed position.

Each valve leaflet 30 may include outer end portions 42 (marked in FIG. 2) that couple to the stent frame 16 of the valve body 10. The outer end portions 42 may couple to the stent frame 16 at commissure points of the leaflets 30 and may pass through openings of the stent frame 16 to couple to the stent frame 16. The coupling may have a variety of forms. For example, each valve leaflet 30 may include tabs 44 at the respective outer end portion 42 of the valve leaflet 30. The tabs 44 may extend through openings in the stent frame 16 to couple to the stent frame 16 and then may be sutured to hold the tabs 44 in position.

Further, each valve leaflet 30 may include a lower end portion 46 that may be sutured to a skirt 50 (marked in FIG. 1) along a scallop line 48 that may comprise a suture line. For example, a lower end portion 46 of each leaflet 30 opposite the upper end portion 32 may be sutured to the skirt 50 (marked in FIG. 1) at a scallop line 48. The sutures of the scallop line 48 may hold the leaflets 30 to the stent frame 16 and prevent undesired fluid flow through the valve body 10 outside of the flow channel 24.

The valve leaflets 30 may be configured to open and close during operation such that the distal end 12 of the valve body 10 forms an outflow end of the valve body 10, and the proximal end 14 of the valve body 10 forms an inflow end of the valve body 10. The valve leaflets 30 may be configured to impede fluid flow in an opposite direction from the outflow end to the inflow end of the valve body 10 when the valve leaflets 30 are in a closed position.

Referring to FIG. 1, the valve body 10 may include one or more skirts 50, 52. The skirt 50 may comprise a skirt that is positioned inward of the stent frame 16 and may be coupled to the plurality of valve leaflets 30 via sutures or another form of coupler. For example, the scallop line 48 may be present on the skirt 50. The skirt 50 may form at least a portion of the inner surface 28 of the valve body 10 and may extend around the entire interior perimeter of the stent frame 16. The skirt 50 in examples may be coupled to the stent frame 16 via sutures or another form of coupler.

The skirt 52 may comprise an outer skirt that may form at least a portion of the outer surface 26 of the valve body 10. The skirt 52 may be positioned radially outward of the one or more prosthetic valve leaflets 30, radially outward of the skirt 50 (which may be considered an inner skirt), and may be positioned radially outward of the stent frame 16 in examples.

The skirt 52 may extend circumferentially about the valve body 10, and may extend circumferentially around the entirety of the valve body 10. The skirt 52 may cover an outer surface of the stent frame 16 at a proximal portion of the stent frame 16, and may extend for only a portion of the axial length of the stent frame 16 (e.g., a proximal portion), or may extend for the entire axial length of the stent frame 16. The skirt 52 may be coupled to the stent frame 16 via sutures or another form of coupler, which may thus couple the skirt 52 to the other components of the valve body 10 (including the valve leaflets 30 and the skirt 50).

FIG. 2 illustrates a top view of the valve body 10 with the valve leaflets 30 in an opened configuration. FIG. 3 illustrates a top view of the valve body 10 with the valve leaflets 30 in a closed configuration.

In examples, the valve body 10 may comprise an implantable prosthetic valve that may be deployed to a heart valve. The prosthetic valve, for example, may comprise a prosthetic aortic heart valve that may be deployed to a native aortic heart valve. The outer surface 26 of the valve body 10, for example, may be configured to apply a force to an annulus of a native aortic valve to anchor the valve body 10 in position. The skirt 52 may contact and press against an inner surface of the native aortic valve to seal with the inner surface of the native aortic valve. The prosthetic valve leaflets 30 may replace the operation of the native aortic valve. The valve body 10 may have a cylindrical shape that may conform to a shape of a native aortic valve. In examples, other forms of valve bodies may be utilized as desired. The valve bodies may be configured to support one or more prosthetic valve leaflets.

The valve body 10 may be deployed to a native mitral or tricuspid valve in examples. Features of the valve body 10, however, may result in use of another body for deployment to a native mitral or tricuspid valve. For example, a shape of a native mitral or tricuspid valve may be irregular or may be non-circular (e.g., a “D” shape or other shape). Deployment to a native mitral or tricuspid valve may pose difficulties due to the compliance of the heart valve annulus and the electrically conductive structures that may be proximate to the annulus.

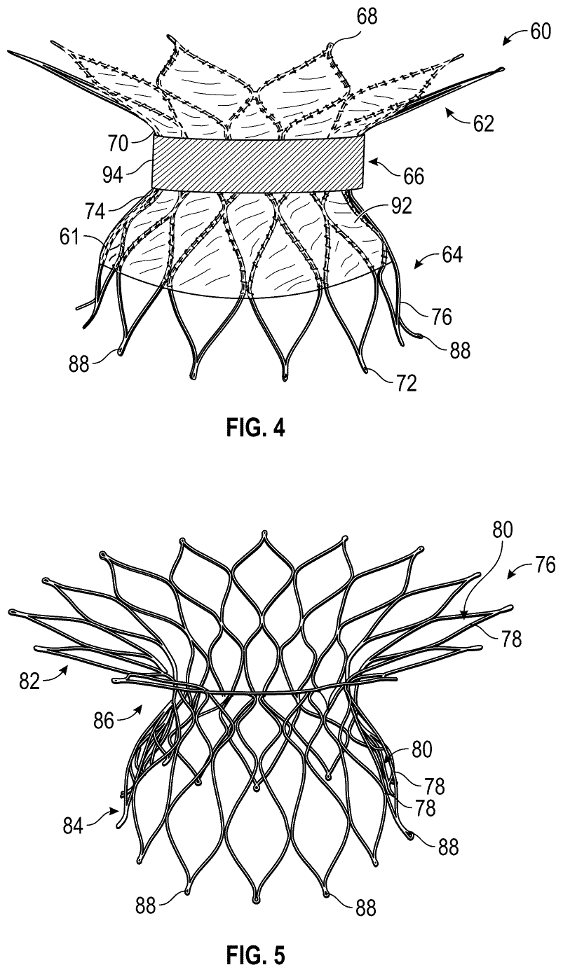

FIG. 4 illustrates a perspective view of a self-expanding stent 60 that may be utilized in examples herein. The stent 60 may be utilized with the valve body 10 to anchor the valve body 10 to a native valve, such as a native mitral or tricuspid valve. The stent 60 may be utilized to anchor the valve body to another form of valve in examples. The stent 60 may comprise an anchoring body or a docking body in examples. The stent 60 may comprise an adaptor configured to adapt the valve body 10 to be deployed to the implantation site. The stent 60 may surround an interior cavity 65 (marked in FIG. 11). The interior cavity 65 may be configured to receive the valve body 10 in examples.

The stent 60 may include an outer surface 61 and an inner surface 63 (marked in FIG. 11) configured to face opposite the outer surface 61. The outer surface 61 may face radially outward from the inner surface 63. The inner surface 63 may face towards the interior cavity 65 of the stent 60 configured to receive the valve body 10. The outer surface 61 may face radially outward towards the native valve annulus (e.g., the native mitral or tricuspid valve annulus).

The stent 60 may include a proximal portion 62, a distal portion 64, and a central portion 66. The central portion 66 may have a smaller diameter than the proximal portion 62 and the distal portion 64. The central portion 66 may form a recessed portion of the stent 60 that may be positioned radially inward of the proximal portion 62 and the distal portion 64. The recessed portion may be configured to be positioned at the annulus of the native heart valve (e.g., the mitral valve annulus or the tricuspid valve annulus). The central portion 66 may comprise a waist portion of the stent 60 that may have a diameter that is less than a diameter of the proximal portion 62 and the diameter of the distal portion 64.

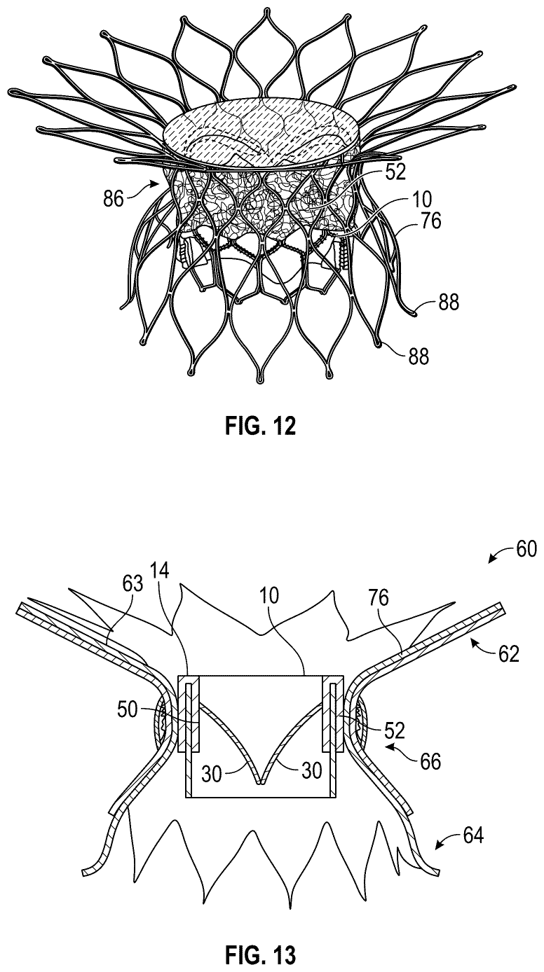

The central portion 66 may be configured for the valve body 10 to dock with. The lesser diameter of the central portion 66, for example, may allow the valve body 10 to expand radially outward to a lesser diameter than the diameter of the respective proximal portion 62 or distal portion 64. The valve body 10, for example, may expand to dock with the central portion 66 as represented in FIGS. 12 and 13. The valve body 10 may contact and press against the inner surface 63 of the stent 60 at the central portion 66.

Referring to FIG. 11, the central portion 66 may have a uniform diameter in examples. The central portion 66 may have a cylindrical shape with a diameter (in the radial dimension 67 of the stent 60) that is uniform along the length of the longitudinal axis 69 of the stent 60 (or the axial dimension). The central portion 66 may have a length along the longitudinal axis 69 at the uniform diameter. The uniform diameter may allow for a uniform cylindrical surface for the valve body 10 to dock with. Other configurations may be utilized in examples.

The proximal portion 62 may comprise a flange extending radially outward from the central portion 66.

The proximal portion 62 may extend radially outward from the central portion 66 in a direction towards a proximal end 68 of the stent 60. The proximal portion 62, for example, may have a greater diameter at the proximal end 68 of the stent 60 than at a coupling portion 70 (marked in FIG. 4) of the proximal portion 62 that couples to the central portion 66. The proximal end 68 or proximal tip of the stent 60 may extend radially outward in a proximal direction.

The proximal portion 62 may have a conical shape from the central portion 66 to a proximal end 68 of the stent 60. The conical shape may comprise a conical frustum shape. The proximal portion 62 may extend circumferentially about a portion of the interior cavity 65 that is surrounded by the proximal portion 62.

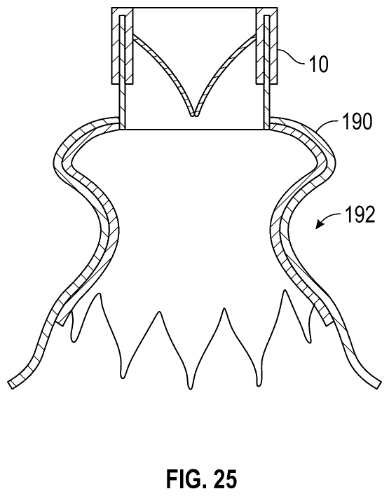

The proximal portion 62 may continue to extend radially outward from the coupling portion 70 to the proximal end 68 or proximal tip of the stent 60. The proximal portion 62 may extend linearly without bends in the proximal portion 62 as the proximal portion extends radially outward. For example, in a side profile as shown in FIG. 11, the proximal portion 62 may extend linearly radially outward from the longitudinal axis 69. The diameter of the proximal portion 62 in the radial dimension 67 may increase linearly in a direction along the longitudinal axis 69. The proximal portion 62 may extend at a single angle as represented in FIG. 11. The single angle may extend from the central portion 66 to the proximal end 68 of the stent 60. The portion of the interior cavity 65 that is surrounded by the proximal portion 62 may have a corresponding shape. Other configurations or shapes of proximal portions 62 may be utilized. In some examples, the proximal portions 62 of the stent 60 can include one or more linear portions and/or one or more curved portions. For example, FIG. 25 illustrates an example in which a proximal portion includes a curved bulb shape.

The proximal portion 62 may be configured to be positioned on an atrial side of the native heart valve in a deployment to a mitral or tricuspid valve. The proximal portion 62 may be configured to contact a surface of the atrium. The proximal portion 62 may resist distal or ventricular movement of the stent 60 (for example, movement of the stent 60 towards the ventricle) and accordingly resist such movement of the valve body 10 coupled to the stent 60. The shape of the proximal portion 62 may beneficially allow the proximal portion 62 to seat upon or rest against the surface of the atrium, and the interaction between the surface of the atrium and the stent 60 may resist distal or ventricular movement of the stent 60.

Referring to FIG. 4, the distal portion 64 may extend radially outward from the central portion 66 in a direction towards a distal end 72 of the stent 60. The distal portion 64, for example, may have a greater diameter at the distal end 72 of the stent 60 than at a coupling portion 74 of the distal portion 64 that couples to the central portion 66. The distal end 72 or distal tip of the stent 60 may extend radially outward in a distal direction. The distal portion 64 may continue to extend radially outward from the coupling portion 74 to the distal end 72 or distal tip of the stent 60.

The distal portion 64 may have a curvature and may bow radially outward as shown in FIGS. 4 and 11. The distal portion 64 may have a curved bulb shape, or other shape as desired. For example, in a side profile as shown in FIG. 11, the distal portion 64 may have a varying and non-linear increase in diameter radially outward from the longitudinal axis 69. The distal portion 64 may have one or more bends 75 forming a plurality of angles of the distal portion 64. An inward portion 77 (more proximate to the central portion 66) may have a different angle in a side profile than an outward portion 79 (further from the central portion 66). The inward portion 77 may have a steeper angle relative to the longitudinal axis 69 than the outward portion 79. A plurality of variations in angle may be provided. The portion of the interior cavity 65 that is surrounded by the distal portion 64 may have a corresponding shape (for example, along the longitudinal axis of the stent 60). Other configurations of distal portions 64 may be utilized.

The distal portion 64 may be configured to be positioned on a ventricular side of the native heart valve in a deployment to a mitral or tricuspid valve. The distal portion 64 may be configured to contact one or more leaflets of a native valve or another ventricular portion of the native heart valve. The distal portion 64 may resist proximal or atrial movement of the stent 60 (e.g., movement towards the aorta) and accordingly resist such movement of the valve body 10 coupled to the stent 60. The shape of the distal portion 64 may beneficially allow the native valve leaflets to be positioned upon the outer surface of the distal portion 64.

The stent 60 may comprise a stent frame 76. FIG. 5, for example, illustrates a perspective view of the stent frame 76 with other features of the stent 60 excluded from view. The stent frame 76 may have a variety of configurations, and may include a plurality of struts 78 separated by spaces 80. The struts 78 may join together at junctures.

The plurality of struts 78 may form a lattice structure for the stent frame 76. The lattice structure may include cells comprising the struts 78 bounding one of the spaces 80. The cells may repeat to form the lattice structure. The cells may have a diamond shape as shown in FIG. 5 or may have another shape as desired.

The configuration of struts 78 separated by spaces 80 may allow the stent frame 76 and accordingly the stent 60 to be compressed into a compressed, undeployed, or unexpanded configuration in examples. For example, the stent 60 may be compressed radially inward into a compressed, undeployed, or unexpanded configuration for deployment. The stent 60 may have a cylindrical shape upon being compressed, with the diameter of the proximal portion 62 being the same as the distal portion 64 and the central portion 66. Alternatively, the diameters of the proximal portion 62, the distal portion 64, and the central portion 66 may be different from each other when compressed (or crimped). A crimping operation may be utilized with the stent 60 to compress the stent frame 76 and the stent 60. The length of the stent frame 76 and stent 60 may be increased and the diameter decreased in such a procedure.

The features of the shape of the stent 60 described herein may apply to the shape of the stent frame 76. The stent frame 76 may be shaped to include the features discussed regarding the stent 60 (e.g., the shape of the corresponding proximal portion 82, distal portion 84, and central portion 86 of the stent frame 76). The shapes of such portions may match the shapes described regarding the stent 60.

The distal portion 84 of the stent frame 76 (and accordingly the distal portion 64 of the stent 60) may include one or more distal prongs 88. The prongs 88 may be configured to engage chordae in a mitral or tricuspid deployment. For example, the prongs 88 may be positioned between chordae or hook chordae as desired. The distal prongs 88 may be positioned at the distal end or tip of the stent 60 in examples. Other positions may be utilized in examples.

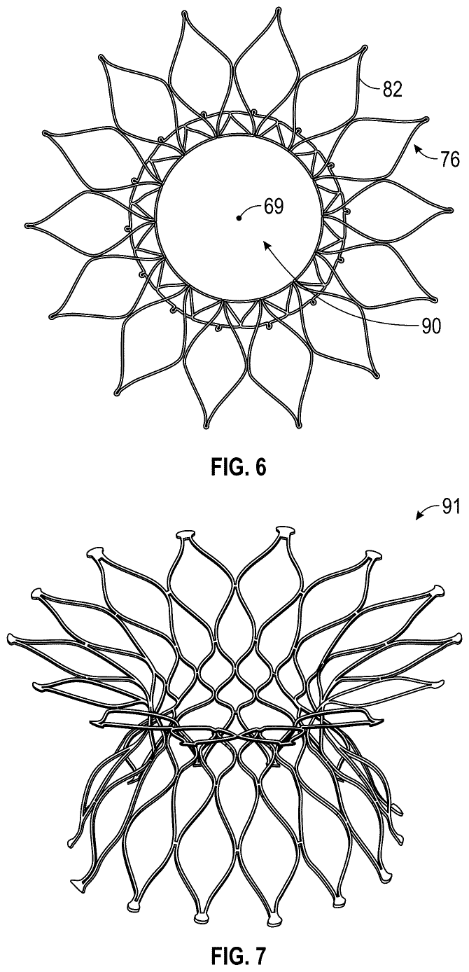

FIG. 6 illustrates a top view or proximal view of the stent frame 76. The interior of the stent frame 76 defining an interior cavity 90 of the stent frame 76 for receiving the valve body 10 is shown.

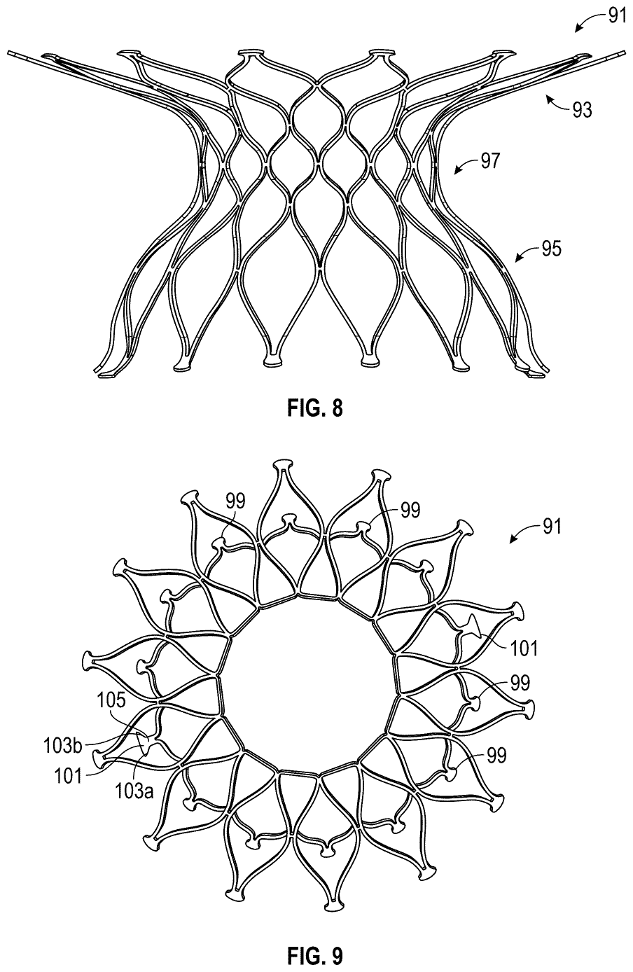

The configuration of the stent frame may vary in examples. FIG. 7, for example, illustrates a perspective view of a stent frame 91 that may be utilized in examples herein. The stent frame 91 may include the features of the stent frame 76 unless stated otherwise. FIG. 8 illustrates a side view of the stent frame 91, with the respective proximal portion 93, distal portion 95, and central portion 97 marked. FIG. 9 illustrates a top view of the stent frame 91.

The stent frame 91 may include one or more distal prongs 99 having flared tips. The size of the flared tips may vary as desired. For example, one or more of the distal prongs may include distal prongs 101 having larger flared tips. The flared tips may protrude circumferentially, and may protrude in opposite directions from each other. For example, each of the distal prongs 101 may have a “T” shape including arms 103a, b and a central support 105. The arms 103a, b may form the cross-bar of the “T” shape, extending transverse to the central support 105. The arms 103a, b may each extend circumferentially away from the central support 105 in opposite directions. The arms 103a, b may be configured to hook around or otherwise engage the chordae. The central support 105 may pass between chordae. Other configurations of distal prongs may be utilized. The distal prongs 99, 101 may be utilized with any example disclosed herein.

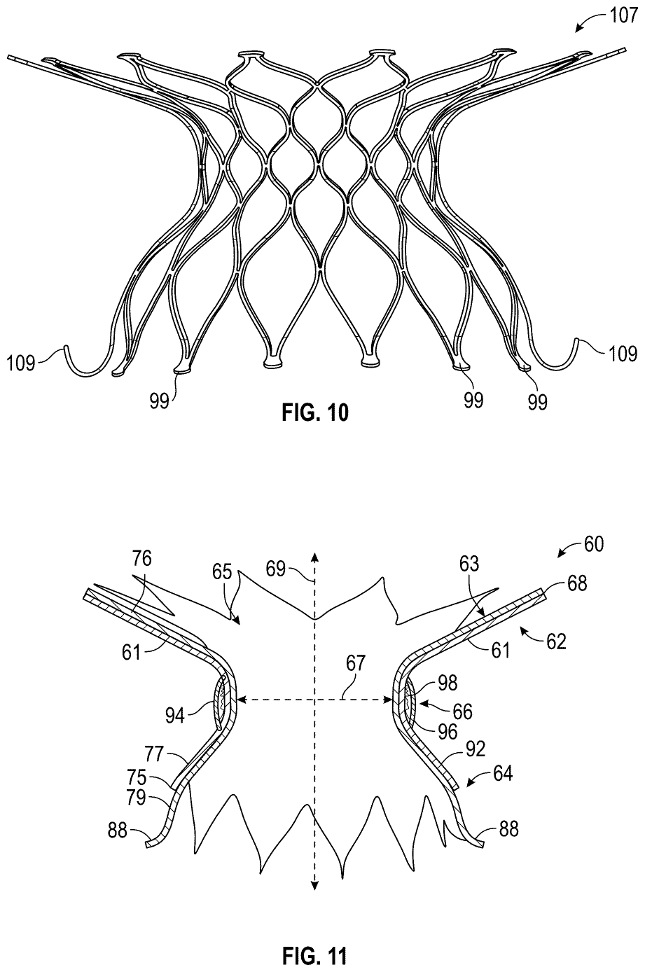

In examples, any or all of the distal prongs may form a hook extending proximally. FIG. 10, for example, illustrates an example of a stent frame 107 including prongs 109 configured similarly as the prongs 101 shown in FIG. 9, yet forming a hook extending proximally. The prongs 109 may comprise a curved hook, with the flared tip of the prong 109 extending proximally. The prongs 109 may be configured to hook around a leaflet of a native valve, with the flared tip of the prong 109 positioned radially outward of a native valve leaflet. The configurations of prongs 109 may be utilized with any example herein. The configurations of the stent frames 91, 107 may be utilized with any example herein.

In examples, a stent may solely comprise a stent frame. In examples, additional features may be provided with a stent. For example, referring to FIG. 4, the stent 60 may include a skirt 92. The skirt 92 may be positioned upon the stent frame 76. For example, the skirt 92 may be positioned on an outer surface of the stent frame 76 (as represented in FIGS. 4 and 11) or may be positioned on an inner surface of the stent frame 76. The skirt 92 may comprise an outer cover of the stent 60. The skirt 92 may be interwoven with struts of the stent frame 76 in examples to be coplanar with the stent frame 76 in examples. Other positions of skirts 92 may be utilized in examples. One or more couplers such as sutures may couple the skirt 92 to the stent frame 76. The skirt 92 may be made of a material such as polyethylene terephthalate (PET) or another form of polymer, or textile, or other material.

The skirt 92 may extend along all or a portion of the proximal portion 62, distal portion 64, or central portion 66 of the stent 60. For example, as shown in FIG. 4, the skirt 92 may extend along the entirety of the proximal portion 62. The skirt 92 may extend to a mid-portion of the distal portion 64. The skirt 92 may partially cover one or more cells of the lattice of the stent frame 76 at the distal portion 64 or may have another configuration as desired. The skirt 92 may be continuous from the proximal portion 62 to the distal portion 64 or may be separated into parts if desired.

The skirt 92 may be configured to reduce fluid flow exterior of the stent 60 (paravalvular leakage). The skirt 92 may comprise a sealing skirt that may be made of a fluid impermeable material. Other configurations of skirts may be utilized in examples.

In examples, the stent 60 may include a sealing band 94. The sealing band 94 may be positioned at the central portion 66 of the stent 60 and may comprise a material configured to form a seal with a native valve annulus or other portion of a native heart valve (e.g., native valve leaflets) as desired. The sealing band 94 may extend circumferentially about the central portion 66 of the stent 60. The sealing band 94 may comprise an annular band and may have a cylindrical shape, which may match a cylindrical shape of the central portion 66 of the stent 60. The sealing band 94 may form the outer surface of the stent 60.

The sealing band 94 may comprise a compressible or compliant material in examples. The sealing band 94 may be configured to be compressed to contour to a shape of the native valve annulus or other portion of a native heart valve.

FIG. 11, for example, illustrates a cross sectional schematic view of the stent 60. The sealing band 94 may include an outer covering 96 that forms a pocket configured to be filled with a filler 98. The outer covering 96 may form the outer surface of the pocket and the skirt 92 may form the inner surface of the pocket. The pocket may surround the stent frame 76. The filler 98 may comprise a compressible material. For example, the filler 98 may comprise a braid material or other form of filler as desired. The braid material may comprise a metal or polymer braid material, or other form of material as desired. The filler 98 may be resilient and configured to expand radially outward to form fit against the annulus.

In example, the sealing band 94 may lack a pocket and may comprise a compliant material forming a band about the central portion 66 of the stent 60. For example, the sealing band 94 may be formed of a braid material that may be positioned at the central portion 66 of the stent 60. Other configurations of sealing bands may be utilized in examples.

The stent 60 may be configured to receive the valve body 10. The valve body 10 may couple to the stent 60. The valve body 10, for example, may dock with the stent 60. The valve body 10 may be positioned within the interior cavity 65 of the stent 60.

FIG. 12, for example, illustrates the valve body 10 docked with the central portion 86 of the stent frame 76 (with other features of the stent 60 excluded from view for clarity). The valve body 10 may couple to the inner surface of the stent 60. In examples, the valve body 10 may press against the inner surface of the stent 60 to dock with the stent 60. The skirt 52 of the valve body 10, for example, may contact and press against the inner surface of the stent 60 and may retain the valve body 10 to the stent 60. The skirt 52 may intermesh with the struts of the stent frame 76 to couple the valve body 10 in position. The valve body 10 may couple to the central portion 66 of the stent 60.

The valve body 10 may dock to the stent 60 with the outlet portion of the valve body 10 extending distally, and the inlet portion of the valve body 10 positioned proximally. Such a configuration may allow for flow in a mitral or tricuspid arrangement, in which flow is allowed in a distal or ventricular direction and impeded in a proximal or atrial direction. The proximal and distal portions of the stent 60 may flare radially outward from the valve body 10.

FIG. 13, for example, illustrates a side cross sectional schematic view of the valve body 10 docked to the stent 60. The valve body 10 may protrude into the portion of the interior cavity surrounded by the distal portion 64 of the stent 60. The proximal end 14 of the valve body 10 may be positioned at the connection of the proximal portion 62 of the stent to the central portion 66.

The stent 60 and particularly the stent frame 76 of the stent 60 may be self-expanding in examples. For example, the stent frame 76 may be made of a shape memory material and may be configured to expand to the expanded, deployed, or uncompressed configuration shown in FIGS. 4-13 for example. The stent frame 76 may be configured to expand from an unexpanded, undeployed, or compressed configuration in which the stent frame 76 has a cylindrical shape along its length. The shape memory material may comprise Nitinol or another form of shape memory material as desired. In examples the stent frame 76 may be self-expanding due to a biasing force present in the material of the stent frame 76.

The stent 60 may comprise a self-expanding stent configured to be deployed to a native heart valve. The stent 60 may beneficially be self-expanding to allow the stent 60 to expand and contour to a shape of an implantation site (e.g., a native mitral or tricuspid valve). The shape of the implantation site may be irregular and a self-expanding stent 60 may be configured to conform to such a shape. Further, a self-expanding stent 60 may provide reduced outward radial force against an implantation site than another form of expansion (e.g., inflatable body expansion), which may reduce the possibility of electrical conduction disturbance with an implantation site. Various other benefits may result.

The valve body 10 may be configured to be expanded with an inflatable body. The valve body 10 may be beneficially expanded with an inflatable body to provide a secure and controlled expansion of the valve body 10 to dock with the stent 60. The stent 60 may provide a uniform docking surface that may allow the valve body 10 to conform to upon expansion of the inflatable body. Other configurations may be utilized in examples.

The valve body 10 and stent 60 may form a system for deployment to a native heart valve. The system may be configured to be deployed to a native mitral heart valve or a native tricuspid valve. Other implantation sites may comprise a pulmonary valve or other implantation site as desired.

Forms of delivery systems may be utilized to deploy one or more of the valve body 10 or the stent 60. Delivery systems may be utilized to deploy both the valve body 10 and the stent 60 along an elongate shaft 100 for deployment of such components.

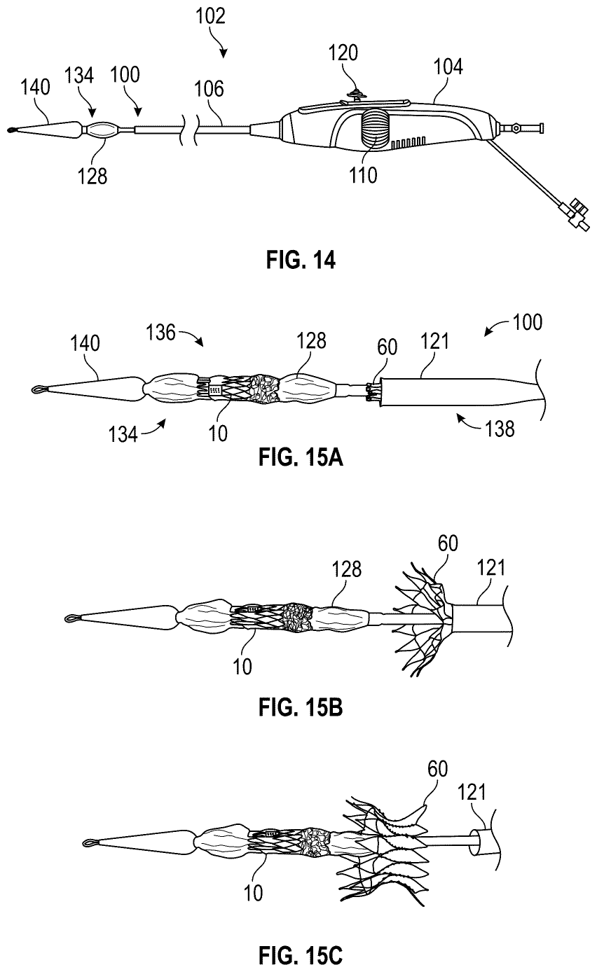

FIG. 14, for example, illustrates a delivery system 102 that may be utilized in examples herein. The delivery system 102 may include an elongate shaft 100 that may include multiple implant retention areas for deployment of the respective valve body 10 and stent 60. The implant retention areas may be positioned in a variety of locations on the elongate shaft 100 as desired.

The delivery system 102 may include a housing 104 or handle at a proximal end portion 106 of the elongate shaft 100. The housing 104 may allow for control of the features of the delivery system 102 as desired.

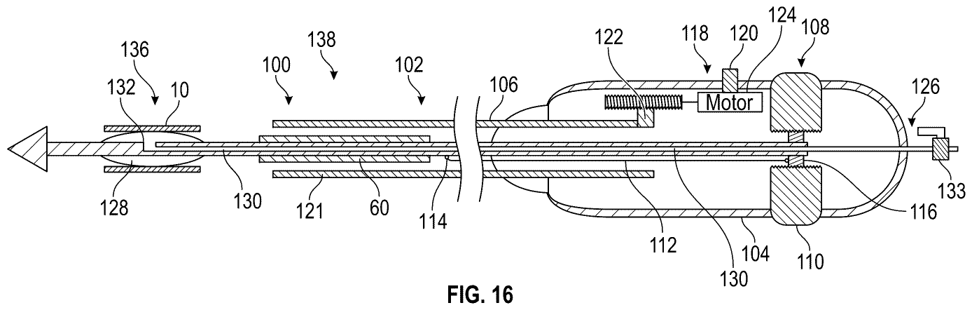

FIG. 16, for example, illustrates a cross sectional view of the delivery system 102. The delivery system 102 may include a deflection mechanism 108 for controlling deflection of the elongate shaft 100. The deflection mechanism 108, for example, may be utilized to cause the elongate shaft 100 to deflect in a direction transverse to the longitudinal extent of the elongate shaft 100. The elongate shaft 100 may deflect in a plane.

The deflection mechanism 108 may include an actuator 110. The actuator 110 may comprise a control knob or other form of actuator (e.g., push button, slide knob, pull trigger, etc.) as desired. The actuator 110 may be configured to control a tether 112. A proximal end portion of the tether 112 may be engaged with the actuator 110. A distal end portion of the tether 112 may couple to a connection point 114 on the elongate shaft 100.

The actuator 110 may be configured to retract or advance the tether 112. The tether 112, for example, may comprise a pull wire that may couple to the connection point 114 on the elongate shaft 100. The tension in the pull wire may be controlled through operation of the actuator 110. The actuator 110 may retract or advance the tether 112 to deflect the elongate shaft 100.

In examples, an adaptor 116 may be utilized that may convert a rotational motion of the actuator 110 to a longitudinal motion of the tether 112. The longitudinal motion of the tether 112 may result in a transverse deflection of the elongate shaft 100. Other forms of deflection mechanisms (e.g., rail systems) may be utilized as desired. One or more bends may be provided in the delivery system 102 to produce a desired movement of the elongate shaft 100. For example, additional tethers may be provided at various locations (e.g., circumferentially offset from the connection point 114 of the tether to the elongate shaft 100) to provide multiple planes of deflection of the elongate shaft 100 as desired.

In examples, the delivery system 102 may include a control mechanism 118 that may be utilized to control a retaining body configured to retain the stent 60 to the elongate shaft 100 in a compressed configuration. The retaining body may comprise a capsule 121, for example, or may have another configuration as desired. The control mechanism 118 may be configured to release the retaining body from the stent 60, for example, by retracting the retaining body from the stent 60.

The control mechanism 118 may include an actuator 120 that may be configured to control the retaining body. The actuator 120 may comprise a push button or may have another form (e.g., a control knob, slide knob, pull trigger, etc.) to control the retaining body.

In examples, the control mechanism 118 may include an adaptor 122 that may be configured to provide longitudinal motion for the retaining body. In examples, the control mechanism 118 may include a motor 124. The motor 124 may be configured to move the retaining body. For example, the motor 124 may move the retaining body in response to input provided by the actuator 120. The motor 124 may move the adaptor 122, for example, by producing rotational motion that is conveyed to the adaptor 122 (via a screw drive or another form of drive). The motor 124 may be configured to retract or advance the retaining body. Other forms of control mechanisms (e.g., non-motorized) may be utilized as desired to control the retaining body.

In examples, the delivery system 102 may include an inflation mechanism 126. The inflation mechanism 126 may be configured to inflate or deflate an inflatable body 128. The inflation mechanism 126, for example, may include a fluid conduit 130. The fluid conduit 130 may extend along the elongate shaft 100 and may be configured for transfer of fluid to or from the inflatable body 128. The elongate shaft 100 may include an opening 132 that may allow for fluid transfer to or from an interior of the inflatable body 128 with the fluid conduit 130. The inflation mechanism 126 may include a valve 133 or port that may control fluid flow through the fluid conduit 130. The valve 133 or port may couple to a fluid reservoir for providing fluid to or from the fluid conduit 130. A pump or other device may be coupled to the valve or port to provide fluid or from the fluid conduit 130. Other forms of inflation mechanisms may be utilized as desired.

FIG. 15A illustrates a close-up view of a distal end portion 134 of the elongate shaft 100. The elongate shaft 100 may include a first implant retention area 136. The first implant retention area 136 may include an inflatable body 128 for an implant in the form of the valve body 10 to be positioned upon. The valve body 10 is shown crimped or otherwise compressed upon the inflatable body 128 in FIG. 15A. The inflatable body 128 may be configured to expand the valve body 10. The inflatable body 128 may be configured to expand to deploy the valve body 10 to the native heart valve from the elongate shaft 100. The inflatable body 128 may comprise a balloon or other form of body for inflating or expanding to accordingly expand the valve body 10. An interior cavity of the inflatable body 128 may be expanded via the fluid conduit 130 in examples. Other forms of inflatable bodies 128 may be utilized in examples.

The stent 60 may be positioned within a second implant retention area 138. The second implant retention area 138 may comprise a retaining body configured to retain the stent 60 to the elongate shaft 100 in a compressed configuration. The retaining body may be in the form of a capsule 121 configured to extend over the stent 60 or may have another configuration as desired (e.g., a band or strip extending over the stent 60). The retaining body may be configured to release from the stent 60 to allow the stent 60 to deploy to the native heart valve from the elongate shaft 100. The capsule 121 may be retracted proximally, for example, to uncover the stent 60. The stent 60 may be allowed to self-expand upon being uncovered by the capsule 121.

The first implant retention area 136 and the second implant retention area 138 may each be positioned at a distal end portion 134 of the elongate shaft 100. Other positions may be utilized as desired.

In a configuration as shown in FIG. 15A, the first implant retention area 136 may be positioned adjacent to the second implant retention area 138. The first implant retention area 136 is positioned distal of the second implant retention area 138, longitudinally adjacent to the second implant retention area 138. As such, the valve body 10 may be positioned upon the elongate shaft 100 distal of the stent 60. Other configurations may be utilized in examples.

A nose cone 140 may be positioned distal of the first implant retention area 136 and may comprise a distal tip of the elongate shaft 100.

FIG. 15B illustrates an exemplary retraction of the capsule 121 to partially expand the stent 60. FIG. 15C illustrates an exemplary retraction of the capsule 121 to allow the stent 60 to fully expand.

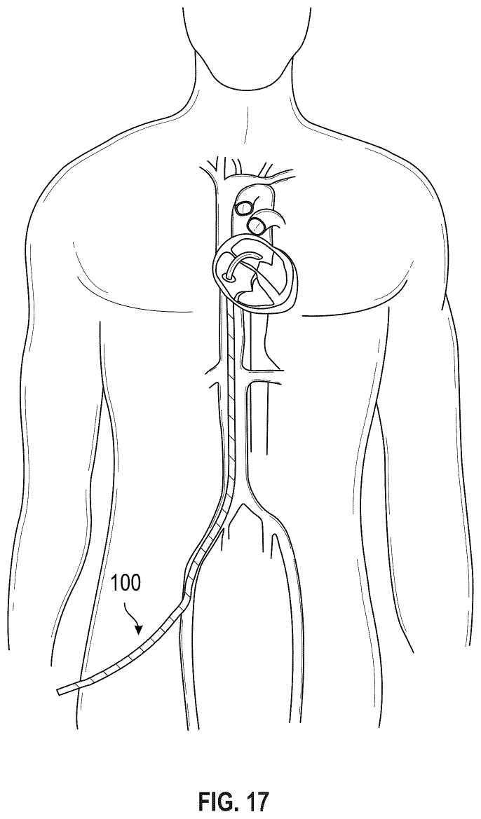

The delivery system 102 may be advanced to a desired implantation site. The delivery system 102 may be advanced through the vasculature of a patient, as represented in FIG. 17 for example. The elongate shaft 100 may be passed percutaneously into the body of the patient. The elongate shaft 100 may be advanced transvenous, for example, to reach a desired implantation site such as the heart. The elongate shaft 100 may be passed transseptal to the left atrium to approach the mitral valve. The elongate shaft 100 may remain in the right atrium to approach the tricuspid valve in examples. Other approaches (e.g., transapical, or others) may be utilized as desired.

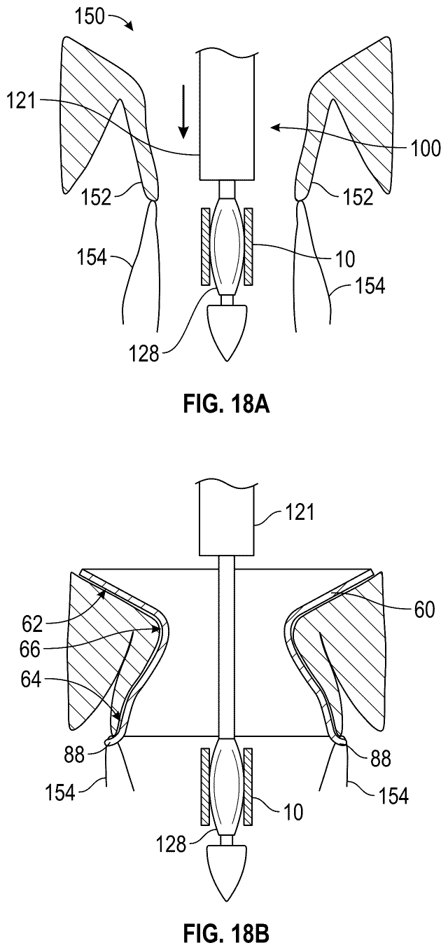

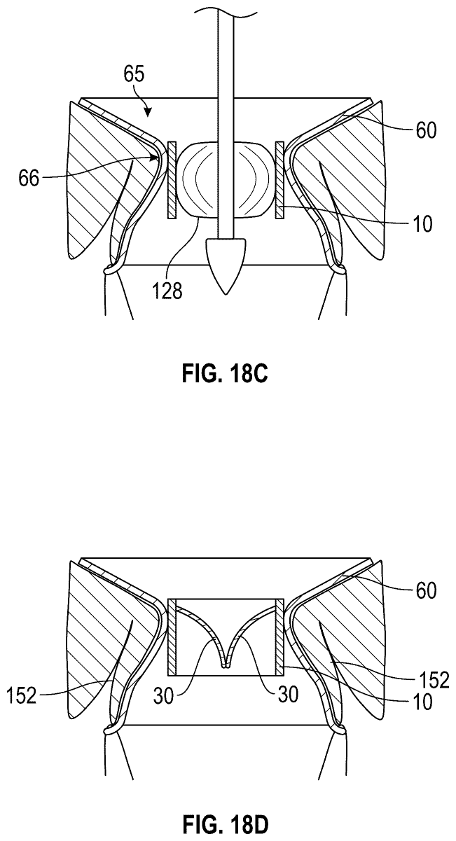

FIGS. 18A-18D illustrate an exemplary deployment sequence of the valve body 10 and stent 60. Referring to FIG. 18A, for example, the elongate shaft 100 may be advanced to an implantation site, here shown as a native mitral valve 150. The native leaflets 152 and connected chordae 154 are shown.

The elongate shaft 100 may be advanced such that the stent 60 covered by the capsule 121 may be positioned at a desired orientation relative to the native valve 150 for deployment.

Upon the stent 60 being placed in the desired orientation, the capsule 121 may be retracted in a manner similar to shown in FIGS. 15B and 15C. The capsule 121 may be retracted from the stent 60 to release from the stent 60. The retraction of the capsule 121 may allow the stent 60 to self-expand as represented in FIG. 18B. The capsule 121 may release from the stent 60 to allow the stent 60 to deploy to the heart valve. The central portion 66 of the stent 60 may be positioned at the valve annulus. The proximal portion 62 of the stent 60 may be positioned on the proximal or atrial side of the valve annulus. The distal portion 64 of the stent 60 may be positioned on the distal or ventricular side of the valve annulus. The distal prongs 88 may engage the chordae 154. The shapes of the proximal portion 62 and distal portion 64 may serve to resist respective distal or proximal forces upon the stent 60.

Referring to FIG. 18C, the elongate shaft 100 and correspondingly the valve body 10 may be retracted proximally to position the valve body 10 as desired relative to the stent 60. The valve body 10 may be positioned within the interior cavity 65 of the stent 60. The valve body 10 may be positioned at the central portion 66 of the stent 60, for example. The inflatable body 128 may be filled with fluid to expand the inflatable body 128. The inflatable body 128 may be expanded to press the valve body 10 radially outward and dock the valve body 10 to the stent 60. The valve body 10 may couple to the stent 60. The valve body 10 may be pressed against the inner surface of the stent 60 with the inflatable body 128 to dock with the stent 60.

A confirmation may be made that the valve body 10 is expanded fully and anchored to the desired portion of the stent 60. The inflatable body 128 may be deflated or contracted and retracted from the valve body 10 as represented in FIG. 18D for example. The prosthetic valve leaflets 30 may be positioned to replace the operation of the native valve leaflets 152.

Other configurations of delivery systems and other forms of deployment sequences may be utilized in examples.

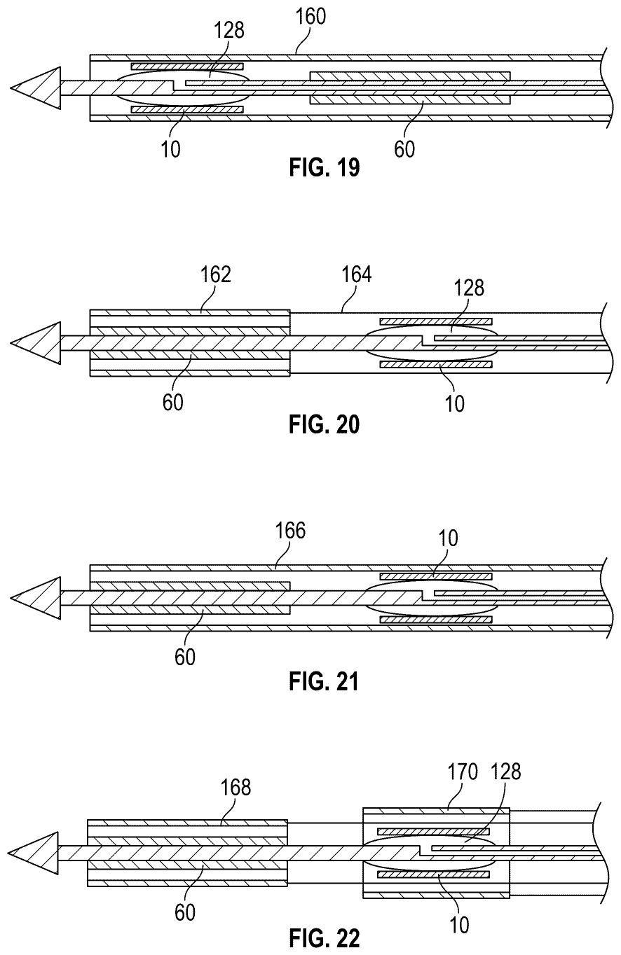

For example, referring to FIG. 19, in examples, a capsule 160 may extend over both the stent 60 and the valve body 10. A single capsule 160, for example, may be utilized extending over both implants. The capsule 160 may extend over the inflatable body 128. A control mechanism, similar to control mechanism disclosed herein may be utilized to retract the capsule 160.

Referring to FIG. 20, in examples, a capsule 162 may extend over the stent 60 yet not the valve body 10. For example, a tether 164 or other retraction mechanism may be utilized to retract the capsule 162 without the capsule 162 extending over the valve body 10. A control mechanism, similar to control mechanism disclosed herein may be utilized to retract the capsule 162.

In examples, the stent 60 may be positioned distal of the valve body 10 as shown in FIG. 20 for example. In such a configuration, the stent 60 may be deployed and then the valve body 10 may be advanced distally to deploy to the stent 60. The second implant retention area accordingly may be positioned distal of the first implant retention area in examples.

FIG. 21 illustrates a configuration in which the stent 60 is positioned distal of the valve body 10. A single capsule 166 may extend over both the stent 60 and the valve body 10. A control mechanism, similar to control mechanism disclosed herein may be utilized to retract the capsule 166.

FIG. 22 illustrates a configuration in which separate capsules 168, 170 may extend over the respective stent 60 and the valve body 10. The capsule 170 may be configured to extend over the valve body 10 positioned upon the inflatable body 128 and release from the valve body 10. In examples, the capsules 168, 170 may be independently controllable. Separate control mechanisms may be utilized to independently deploy the stent 60 or valve body 10 as desired. The control mechanisms may be similar in configuration to control mechanisms disclosed herein. The capsule 168 may be initially retracted proximally. The capsule 168 may retract and pass proximally through the space or gap positioned between the valve 10 and the capsule 170 (e.g., between the interior surface of the capsule 170 and the exterior surface of the valve 10). The capsule 170 may then be retracted proximally for deployment of the valve 10. Other configurations may be utilized in examples.

A gap between the capsules 168, 170 may reduce the rigidity of the elongate shaft and improve flexibility of the elongate shaft.

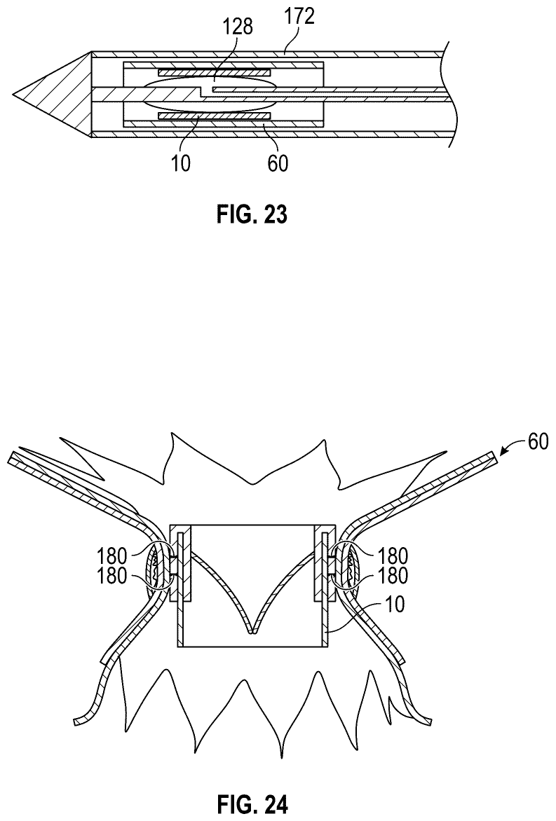

FIG. 23 illustrates a configuration in which the stent 60 may be positioned upon the valve body 10 in a crimped, compressed, or unexpanded configuration. The stent 60 may be positioned radially outward of the valve body 10 upon approach to the implantation site. The capsule 172 may be retracted to allow the stent 60 to expand. The inflatable body 128 may then be inflated to expand the valve body 10.

FIG. 24 illustrates an example in which one or more couplers 180 may be utilized to couple the valve body 10 to the stent 60. The couplers 180, for example, may comprise sutures, fabric, locks, snaps, or other forms of couplers 180. The couplers 180 may extend from the valve body 10 to the stent 60. The valve body 10 may be preattached to the stent 60 upon deployment in such a configuration. A delivery configuration as shown in FIG. 23, for example, may be utilized.

The delivery systems disclosed herein may be utilized solely or in combination with other features disclosed herein. In examples, the delivery systems may be utilized to deploy other forms of implants. The implants disclosed herein may be deployed with other forms of delivery systems in examples.

In examples, the position that the valve body 10 couples to a stent may vary. FIG. 25, for example, illustrates a configuration in which the valve body 10 may be coupled to a proximal end portion 190 of a stent 192. The stent 192 may be configured similarly as the stent 60 discussed herein, yet with the proximal end portion 190 of the stent 192 deflected radially inward to couple to the valve body 10. The valve body 10 may protrude proximally from the stent 192. A skirt may be provided that may seal the interior of the stent 192 and the valve body 10 to reduce fluid flow outside of the combination of the valve body 10 and stent.

Various other configurations may be utilized in examples.

The deployment and delivery apparatuses and systems disclosed herein may be utilized for mitral, tricuspid, aortic, and pulmonary replacement and repair as well. The deployment and delivery apparatuses may comprise deployment and delivery apparatuses for delivery of implants such as stents or filters or diagnostic devices, among others.

The deployment and delivery apparatuses and the systems disclosed herein may be used in transcatheter mitral or tricuspid valve implantation or replacement of other native heart valves (e.g., pulmonary or aortic). The deployment and delivery apparatuses and the systems disclosed herein may be utilized for transarterial access, including transfemoral access, to a patient's heart. The deployment apparatuses and systems may be utilized in transcatheter percutaneous procedures, including transarterial procedures, which may be transfemoral or transjugular. Transapical procedures, among others, may also be utilized. Other procedures may be utilized as desired.

Features of examples may be modified, substituted, excluded, or combined across examples as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A system for deployment to a native heart valve, the system comprising: a self-expanding stent configured to be deployed to the native heart valve and including a proximal portion, a distal portion, and a central portion having a smaller diameter than the proximal portion and the distal portion, the proximal portion comprising a flange extending radially outward from the central portion and having a conical shape from the central portion to a proximal end of the self-expanding stent, the self-expanding stent surrounding an interior cavity; and a valve body configured to be expanded with an inflatable body and configured to be positioned within the interior cavity of the self-expanding stent and couple to the central portion, the valve body including one or more prosthetic valve leaflets.

Example 2: The system of any example herein, in particular Example 1, wherein the distal portion extends radially outward from the central portion in a direction towards a distal end of the self-expanding stent.

Example 3: The system of any example herein, in particular Example 1 or Example 2, wherein the distal portion bows radially outward.

Example 4: The system of any example herein, in particular Examples 1-3, wherein the distal portion has a curved bulb shape.

Example 5: The system of any example herein, in particular Examples 1-4, wherein the central portion has a uniform diameter.

Example 6: The system of any example herein, in particular Examples 1-5, further comprising one or more distal prongs for engaging chordae of a heart.

Example 7: The system of any example herein, in particular Example 6, wherein each of the distal prongs forms a hook extending proximally.

Example 8: The system of any example herein, in particular Examples 1-7, wherein the self-expanding stent includes a stent frame.

Example 9: The system of any example herein, in particular Example 8, wherein the stent frame is made of a shape memory material.

Example 10: The system of any example herein, in particular Examples 1-9, wherein the self-expanding stent includes a skirt.

Example 11: The system of any example herein, in particular Examples 1-10, wherein the self-expanding stent includes a sealing band positioned at the central portion.

Example 12: The system of any example herein, in particular Examples 1-11, wherein the valve body includes a stent frame.

Example 13: The system of any example herein, in particular Example 12, wherein the stent frame of the valve body has a cylindrical shape.

Example 14: The system of any example herein, in particular Examples 1-13, wherein the self-expanding stent has an inner surface and an outer surface facing radially outward from the inner surface, and the valve body is configured to couple to the inner surface.

Example 15: The system of any example herein, in particular Example 14, wherein the valve body includes a skirt configured to contact the inner surface of the self-expanding stent.

Example 16: The system of any example herein, in particular Examples 1-15, wherein the valve body is configured to dock with the self-expanding stent.

Example 17: The system of any example herein, in particular Example 16, wherein the self-expanding stent has an inner surface and an outer surface facing radially outward from the inner surface, and the valve body is configured to press against the inner surface to dock with the self-expanding stent.

Example 18: The system of any example herein, in particular Examples 1-17, further comprising one or more couplers coupling the valve body to the self-expanding stent.

Example 19: The system of any example herein, in particular Examples 1-18, wherein the valve body comprises a prosthetic aortic heart valve.

Example 20: The system of any example herein, in particular Examples 1-19, wherein the system is configured to be deployed to a native mitral heart valve or a native tricuspid heart valve.

Example 21: A delivery system for deployment of a prosthetic system to a native heart valve, the delivery system comprising: an elongate shaft including: a first implant retention area comprising an inflatable body for a first implant to be positioned upon, the inflatable body configured to expand to deploy the first implant to the native heart valve from the elongate shaft, and a second implant retention area comprising a retaining body configured to retain a second self-expanding implant to the elongate shaft in a compressed configuration, the retaining body configured to release from the second self-expanding implant to allow the second self-expanding implant to deploy to the native heart valve from the elongate shaft.

Example 22: The delivery system of any example herein, in particular Example 21, further comprising a fluid conduit extending along the elongate shaft for transfer of fluid to or from the inflatable body.

Example 23: The delivery system of any example herein, in particular Example 21 or Example 22, further comprising a control mechanism for releasing the retaining body from the second self-expanding implant.

Example 24: The delivery system of any example herein, in particular Examples 21-23, wherein the first implant retention area is positioned adjacent to the second implant retention area.

Example 25: The delivery system of any example herein, in particular Examples 21-24, wherein the first implant retention area is positioned distal of the second implant retention area.

Example 26: The delivery system of any example herein, in particular Examples 21-24, wherein the second implant retention area is positioned distal of the first implant retention area.

Example 27: The delivery system of any example herein, in particular Examples 21-26, wherein the retaining body comprises a capsule configured to extend over the second self-expanding implant.

Example 28: The delivery system of any example herein, in particular Example 27, wherein the capsule is configured to extend over the inflatable body.

Example 29: The delivery system of any example herein, in particular Example 27 or Example 28, wherein the capsule is a first capsule, and further comprising a second capsule configured to extend over the first implant positioned upon the inflatable body and release from the first implant.

Example 30: The delivery system of any example herein, in particular Example 29, wherein the first capsule and the second capsule are independently controllable.

Example 31: The delivery system of any example herein, in particular Examples 21-30, wherein the elongate shaft includes a proximal end portion and a distal end portion, and the first implant retention area and the second implant retention area are each positioned at the distal end portion of the elongate shaft.

Example 32: The delivery system of any example herein, in particular Example 31, further comprising a handle positioned at the proximal end portion of the elongate shaft.

Example 33: The delivery system of any example herein, in particular Examples 21-32, wherein the inflatable body is configured to expand to dock the first implant to the second self-expanding implant.

Example 34: The delivery system of any example herein, in particular Examples 21-33, wherein the delivery system is configured to extend through the vasculature of a patient to approach a native mitral valve or a native tricuspid valve.

Example 35: The delivery system of any example herein, in particular Examples 21-34, further comprising the first implant and the second self-expanding implant, wherein the first implant comprises a valve body having one or more prosthetic valve leaflets, and the second self-expanding implant comprises a self-expanding stent.

Example 36: A method comprising: deploying a prosthetic system to a native heart valve, the prosthetic system including: a self-expanding stent including a proximal portion, a distal portion, and a central portion having a smaller diameter than the proximal portion and the distal portion, the proximal portion comprising a flange extending radially outward from the central portion and having a conical shape from the central portion to a proximal end of the self-expanding stent, the self-expanding stent surrounding an interior cavity, and a valve body configured to be expanded with an inflatable body and configured to be positioned within the interior cavity of the self-expanding stent and couple to the central portion, the valve body including one or more prosthetic valve leaflets.

Example 37: The method of any example herein, in particular Example 36, wherein the distal portion extends radially outward from the central portion in a direction towards a distal end of the self-expanding stent.

Example 38: The method of any example herein, in particular Example 36 or Example 37, wherein the distal portion has a curved bulb shape.

Example 39: The method of any example herein, in particular Examples 36-38, wherein the self-expanding stent includes one or more distal prongs for engaging chordae of a heart.

Example 40: The method of any example herein, in particular Examples 36-39, wherein the self-expanding stent includes a sealing band positioned at the central portion.

Example 41: The method of any example herein, in particular Examples 36-40, wherein the valve body includes a stent frame.

Example 42: The method of any example herein, in particular Examples 36-41, wherein the self-expanding stent has an inner surface and an outer surface facing radially outward from the inner surface, and the valve body is configured to couple to the inner surface.

Example 43: The method of any example herein, in particular Examples 36-42, further comprising docking the valve body to the self-expanding stent.

Example 44: The method of any example herein, in particular Examples 36-43, wherein the self-expanding stent has an inner surface and an outer surface facing radially outward from the inner surface, and the method further comprises pressing the valve body against the inner surface with the inflatable body to dock with the self-expanding stent.

Example 45: The method of any example herein, in particular Examples 36-44, further comprising utilizing a delivery system to deploy the self-expanding stent and the valve body, the delivery system including an elongate shaft having: a first implant retention area comprising the inflatable body for the valve body to be positioned upon, and a second implant retention area comprising a retaining body configured to retain the self-expanding stent to the elongate shaft in a compressed configuration.

Example 46: The method of any example herein, in particular Example 45, further comprising releasing the retaining body from the self-expanding stent to allow the self-expanding stent to deploy to the native heart valve.

Example 47: The method of any example herein, in particular Example 45 or Example 46, further comprising advancing the delivery system distally or retracting the delivery system proximally to position the valve body within the interior cavity of the self-expanding stent.

Example 48: The method of any example herein, in particular Examples 36-47, wherein one or more couplers couple the valve body to the self-expanding stent.

Example 49: The method of any example herein, in particular Examples 36-48, wherein the valve body comprises a prosthetic aortic heart valve.

Example 50: The method of any example herein, in particular Examples 36-49, wherein the native heart valve is a native mitral valve or a native tricuspid valve.

Example 51: A method comprising: deploying a prosthetic system to a native heart valve utilizing a delivery system, the prosthetic system including a first implant and a second self-expanding implant, the delivery system including an elongate shaft having: a first implant retention area comprising an inflatable body for the first implant to be positioned upon, the inflatable body configured to expand to deploy the first implant to the native heart valve from the elongate shaft, and a second implant retention area comprising a retaining body configured to retain the second self-expanding implant to the elongate shaft in a compressed configuration, the retaining body configured to release from the second self-expanding implant to allow the second self-expanding implant to deploy to the native heart valve from the elongate shaft.

Example 52: The method of any example herein, in particular Example 51, further comprising filling the inflatable body with fluid to expand the inflatable body.

Example 53: The method of any example herein, in particular Example 51 or Example 52, further comprising retracting the retaining body from the second self-expanding implant to release from the second self-expanding implant.

Example 54: The method of any example herein, in particular Examples 51-53, wherein the first implant retention area is positioned adjacent to the second implant retention area.

Example 55: The method of any example herein, in particular Examples 51-54, wherein the first implant retention area is positioned distal of the second implant retention area.

Example 56: The method of any example herein, in particular Examples 51-54, wherein the second implant retention area is positioned distal of the first implant retention area.

Example 57: The method of any example herein, in particular Examples 51-56, wherein the retaining body comprises a capsule configured to extend over the second self-expanding implant.

Example 58: The method of any example herein, in particular Example 57, wherein the capsule is configured to extend over the inflatable body.

Example 59: The method of any example herein, in particular Example 57 or Example 58, wherein the capsule is a first capsule, and further comprising a second capsule configured to extend over the first implant positioned upon the inflatable body and release from the first implant.

Example 60: The method of any example herein, in particular Example 59, wherein the first capsule and the second capsule are independently controllable.

Example 61: The method of any example herein, in particular Examples 51-60, further comprising docking the second self-expanding implant with the first implant.

Example 62: The method of any example herein, in particular Examples 51-61, wherein the first implant comprises a valve body having one or more prosthetic valve leaflets, and the second self-expanding implant comprises a self-expanding stent.

Example 63: The method of any example herein, in particular Example 62, further comprising advancing the delivery system distally or retracting the delivery system proximally to position the valve body within an interior cavity of the self-expanding stent.

Example 64: The method of any example herein, in particular Examples 51-63, wherein the first implant is coupled to the second self-expanding implant with one or more couplers.

Example 65: The method of any example herein, in particular Examples 51-64, wherein the native heart valve is a native mitral valve or a native tricuspid valve.

Any of the features of any of the examples, including but not limited to any of the first through 65 examples referred to above, is applicable to all other aspects and examples identified herein, including but not limited to any examples of any of the first through 65 examples referred to above. Moreover, any of the features of an example of the various examples, including but not limited to any examples of any of the first through 65 examples referred to above, is independently combinable, partly or wholly with other examples described herein in any way, e.g., one, two, or three or more examples may be combinable in whole or in part. Further, any of the features of the various examples, including but not limited to any examples of any of the first through 65 examples referred to above, may be made optional to other examples. Any example of a method can be performed by a system or apparatus of another example, and any aspect or example of a system or apparatus can be configured to perform a method of another aspect or example, including but not limited to any examples of any of the first through 65 examples referred to above.

The features of the examples disclosed herein may be implemented independently, or independent of other components disclosed herein. The various apparatuses of the system may be implemented independently.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system for deployment to a native heart valve, the system comprising:
a self-expanding stent configured to be deployed to the native heart valve and including a proximal portion, a distal portion, and a central portion having a smaller diameter than the proximal portion and the distal portion, the proximal portion comprising a flange extending radially outward from the central portion and having a conical shape from the central portion to a proximal end of the self-expanding stent, the self-expanding stent surrounding an interior cavity; and
a valve body configured to be expanded with an inflatable body and configured to be positioned within the interior cavity of the self-expanding stent and couple to the central portion, the valve body including one or more prosthetic valve leaflets.

2. The system of claim 1, wherein the distal portion extends radially outward from the central portion in a direction towards a distal end of the self-expanding stent.

3. The system of claim 1, wherein the distal portion bows radially outward.

4. The system of claim 1, wherein the distal portion has a curved bulb shape.

5. The system of claim 1, wherein the central portion has a uniform diameter.

6. The system of claim 1, further comprising one or more distal prongs for engaging chordae of a heart.

7. The system of claim 6, wherein each of the distal prongs forms a hook extending proximally.

8. The system of claim 1, wherein the self-expanding stent includes a stent frame.

9. The system of claim 8, wherein the stent frame is made of a shape memory material.

10. The system of claim 1, wherein the self-expanding stent includes a skirt.

11. The system of claim 1, wherein the self-expanding stent includes a sealing band positioned at the central portion.

12. The system of claim 1, wherein the valve body includes a stent frame.

13. The system of claim 12, wherein the stent frame of the valve body has a cylindrical shape.

14. The system of claim 1, wherein the self-expanding stent has an inner surface and an outer surface facing radially outward from the inner surface, and the valve body is configured to couple to the inner surface.

15. The system of claim 14, wherein the valve body includes a skirt configured to contact the inner surface of the self-expanding stent.

16. The system of claim 1, wherein the valve body is configured to dock with the self-expanding stent.

17. The system of claim 16, wherein the self-expanding stent has an inner surface and an outer surface facing radially outward from the inner surface, and the valve body is configured to press against the inner surface to dock with the self-expanding stent.

18. The system of claim 1, further comprising one or more couplers coupling the valve body to the self-expanding stent.

19. The system of claim 1, wherein the valve body comprises a prosthetic aortic heart valve.

20. The system of claim 1, wherein the system is configured to be deployed to a native mitral heart valve or a native tricuspid heart valve.
